(19) United States
(12) Patent Application Publication
(10) Pub. No.: US 2022/0355022 A1
(43) Pub. Date: Nov. 10, 2022

(54) STEPLESS SPEED REGULATION MEDICAL PULSE IRRIGATOR WITH LED LAMP

(71) Applicant: Apex (Guangzhou) Tools & Orthopedics Co.,ltd., Guangzhou (CN)

(72) Inventors: Jiangxin DU, Guangzhou (CN); Shenghu LIANG, Guangzhou (CN); Chan MOU, Guangzhou (CN); Xianwei ZENG, Guangzhou (CN)

(21) Appl. No.: 17/307,872

(22) Filed: May 4, 2021

(51) Int. Cl. A61M 3/02

(52) U.S. Cl. CPC A61M 3/0287; A61M 3/0216; A61M 3/022; A61M 3/0275; A61M 3/0258; A61M 2205/587

(57) ABSTRACT

Disclosed is a disposable stepless speed regulation medical pulse irrigator with an LED lamp, including an upper handle, a lower handle, a pump body, a fluid tube, stepless speed regulation control parts, a spray nozzle part, a power supply part and an LED lamp. The pump body is arranged inside the upper handle; the left side of the pump body is connected with a piston; and the bottom end of the piston is provided with a big gear. In a pulse irrigating process of a wound, the disposable stepless speed regulation medical pulse irrigator with the LED lamp can use a continuous regulatable flushing speed and pressure to reduce the discomfort of a patient with this instrument according to the wound pain sensitivity of the patient, and can improve the wound cleansing efficiency and relieve the working load of medical staff at the same time. When flushing fluid splashes onto a contaminated fluid collection bag to obstruct the field of view, the LED lamp can be turned on through an LED lamp control switch to assist in the observation for the wound, which facilitates debridement personnel to determine whether debridement is completed, so that the debridement time and cost are greatly saved.

### TECHNICAL FIELD

The present disclosure relates to the technical field of pulse irrigating devices, and more particularly relates to a stepless speed regulation medical pulse irrigator with an LED lamp.

### BACKGROUND

A medical pulse irrigator is a debriding device that provides pulse flushing. When foreign bodies, tissue debris, congestion, etc. in a wound are thoroughly eliminated, a secondary injury to surrounding normal tissues is avoided, and the risk of occurrence of cross infection is lowered. A medical pulse debridement irrigator is generally composed of a handle, a spray nozzle and a battery pack. During use, the handle is held and aligned with a wound surface for flushing. The medical pulse irrigator is one of commonly used instruments for acute and chronic wound debriding.

At present, there have been similar irrigators on the market, but they have a monotonous irrigating speed and extremely high pressure, so that some patients sensitive to pain of wounds are reluctant to use such an instrument, and traditional low-efficiency and high-working-intensity methods such as a manual injector are still used for debriding. Moreover, when an irrigator is used for irrigating, flushing fluid is likely to splash onto a contaminated fluid collection bag, resulting in obstruction to a field of view and inconvenience in debriding.

It is under this background that the present disclosure is proposed. In a wound debriding process, this disposable stepless speed regulation medical pulse irrigator with an LED lamp can use a steplessly regulatable irrigating speed and pressure to reduce the discomfort of a patient with this instrument according to the wound pain sensitivity of the patient, and can improve the wound debriding efficiency and relieve the workload of medical staffs at the same time. When the irrigating fluid splashes onto the contaminated fluid collection bag to obstruct the field of view, the LED lamp can be turned on through an LED lamp control switch to assist in the observation for the wound, which facilitates debridement personnel to determine whether debridement is thoroughly completed, so that the debridement time and cost are greatly saved.

### SUMMARY

The present disclosure has been developed to provide a stepless speed regulation medical pulse irrigator with an LED lamp to improve on some shortcomings of existing pulse devices of this general kind on the present market: Due to a monotonous irrigating speed and a relatively high pressure, some patients with pain sensitivity to wounds are reluctant to choose such an instrument, and instead choose traditional low-efficiency and high-working-intensity methods such as syrings for cleansing. Moreover, when an irrigator is used for debriding, irrigating fluid is likely to splash onto a contaminated fluid collection bag, resulting in obstruction to observation of debridement.

In order to continuing improve on the shortcomings mentioned above, the present disclosure provides the following technical solution: A stepless speed regulation medical pulse irrigator with an LED lamp includes an upper handle, a lower handle, a pump body, a fluid tube, stepless speed regulation control parts, a spray nozzle part, a power supply part and an LED lamp. The pump body is arranged inside the upper handle; the left side of the pump body is connected with a piston, and the bottom end of the piston is provided with a big gear; a locating pin is arranged in the middle of the bottom end of the big gear, and a motor-gear assembly is arranged below the big gear; the stepless speed regulation control parts are mounted on a side edge and the rear side of the motor-gear assembly; and the stepless speed regulation control part on the rear side of the motor-gear assembly is connected with an LED lamp control switch.

Typically, the stepless speed regulation feature is composed of a stepless speed regulation control panel (6), a tact switch (4), a linear speed regulation switch (5) and the motor-gear assembly (7). The rotation speed of the motor-gear assembly (7) may be steplessly controlled by operating the tact switch (4) and the linear speed regulation switch (5).

Typically, a self-locking structure between the tact switch (4) and the linear speed regulation switch (5): the left view of FIG. 5 illustrates a switch off state or a zero-speed state; the lamp is turned on and the motor-gear assembly enter a standby state after the tact switch (4) is pressed; at this time, the linear speed regulation switch (5) is pushed, and the motor-gear assembly (7) starts to run; the rotation speed of the motor-gear assembly (7) also continuously increases with pushing of the linear speed regulation switch (5) till the switch is pushed to the top, as shown in the right view of FIG. 5, the motor-gear assembly (7) reaches the highest rotation speed; at this time, the tact switch (4) will be non-operational to achieve a self-locking purpose and ensure that the motor-gear assembly (7) runs at a rotation speed from low to high, instead of suddenly initiating the motor-gear assembly (7) at a high speed; this is an effective way to improve the patient's experience of wound debridement.

Typically, the stepless speed regulation may be carried out throughout 0-6 V-12 V, in which steady continuous operation can be realized at any speed. Two reference speeds are preset: a low speed (6 V) and a high speed (12 V). The speeds are shown in FIG. 5. Meanwhile, a stepless speed variation module has an additional function: setting delay power cut-off, working time and numbers, and the like. Therefore, the experience of medical staff is enhanced.

Typically, the tact switch in the stepless speed regulation system is provided with an LED state indication lamp, which is user-friendly and improves the availability.

Typically, the LED lamp is a high-brightness white light or high-brightness warm light, and a pump housing assembly is in tight fit with the left side of the outer wall of the upper handle; and the LED lamp, the upper handle and the lower handle are fixedly connected.

Typically, a battery case (28) in a battery pack (16) is provided with a fuse, so that a danger caused by short circuiting of the product can be avoided, and the safety of the product is effectively improved.

Typically, the product is sterilized by an environmentally-friendly efficient sterilization method: plasma sterilization that is short in sterilization time and can effectively improve the production efficiency and reduce the cost; after the sterilization is completed, there is no residue, and the product can be used immediately, which is extremely safe to human and the environment.

Typically, a tube with wire (13) is connected with a handpiece through tube connecting bodies (26, 27) and wire connecting bodies (24, 25). The handpiece and the tube with wire are packaged separately. During use, they are connected together to facilitate sterilization for the product; moreover, mis-initiation of the product during transportation can also be avoided; in this way the safety of the product is improved.

Compared with the related art, the present disclosure has the beneficial effects:

1. In a process of pulse debriding of a wound with the stepless speed regulation medical pulse irrigator with the LED lamp, for some patients sensitive to pain of the wound or having more fragile wound tissues, the rotation speed of a motor can be controlled through the linear stepless switch to adjust the speed and pressure of the flushing fluid to achieve the best irrigating effect.

2. A combined switch structure of the stepless speed regulation medical pulse irrigator with the LED lamp can realize self-locking in the use process to ensure that the irrigating speed is gradually increased from low to high, instead of a sudden increase causing the patient to feel uncomfortable.

3. In the process of pulse debridement with the disposable medical pulse irrigator with the LED lamp, when the irrigating fluid splashes onto the contaminated fluid collection bag to obstruct the observation, the LED lamp can be turned on through an LED lamp control switch to assist in the observation for the wound, which facilitates debridement personnel to determine whether debridement is thoroughly completed, so that the debridement time and cost are greatly saved.

4. The LED lamp is fixed below the handle, connected to the pump housing assembly, and fixed in the lower handle to make the structure stabler, so that the LED lamp cannot completely pass through a through hole of the pump housing assembly and is prevented from falling off, and the firmness of the structure of the device is improved.

5. The battery pack of the product is provided with a fuse, so that a danger caused by short circuiting of the product can be avoided, and the safety of the product is effectively improved.

6. The product is sterilized by plasma sterilization method. Compared with Gamma irradiation sterilization commonly used for existing products of this general kind, the plasma sterilization is short in time, and can effectively improve the production efficiency and reduce the cost; after the sterilization is completed, there is no residue, and the product can be used immediately, which is extremely safe to human and the environment.

7. The tube with wire of the product and the handpiece are of a split type structural design. During packaging, the handpiece and the tube with wire are packaged separately. During use, they are connected together to facilitate sterilization for the product; and moreover, mis-initiation of the product during transportation may also be avoided, which improves the safety of the product.

### BRIEF DESCRIPTION OF DRAWINGS

FIG. 1 is a schematic diagram of an external structure of the disclosure.

FIG. 2 is a schematic diagram of a side structure of the disclosure.

FIG. 3 is a schematic diagram of an LED lamp of the disclosure.

FIG. 4 is a schematic structural diagram of a system composition and a connection relationship of the disclosure.

FIG. 5 illustrates a stepless speed regulation combined switch of the disclosure, wherein the left view illustrates speed 0 or a standby state, and the right view illustrates a high-speed state.

FIG. 6 is a schematic diagram illustrating that a (handpiece) and a fastener of a spray nozzle are self-locked after upper and lower handles are combined according to the present disclosure.

FIG. 7 is a schematic diagram illustrating that a (host) and two passageways of a spray nozzle are sealed after upper and lower handles are combined according to the disclosure.

FIG. 8 is a schematic diagram illustrating that a battery case is provided with a fuse according to the present disclosure.

Specifications of the reference numerals in the drawings: 1: upper handle; 2: lower handle; 3: spray nozzle; 4: tact switch; 5: linear speed regulation switch; 6: speed regulation control panel; 7: motor-gear assembly; 8: pin; 9: big gear; 10: piston O-ring assembly; 11: pump housing assembly; 12: clamp; 13: tube with wire; 14: spike; 15: spike cap; 16: battery pack; 17: sealing ring A; 18: sealing ring B; 19: elastic structure of the spray nozzle; 20: buckle; 21: buckle; 22: LED lamp; 23: LED lamp control switch; 24: wire connecting body B; 25: wire connecting body A; 26: tube connecting body B; 27: tube connecting body A; 28: battery case.

### DETAILED DESCRIPTION

The technical solutions in the embodiments of the present disclosure will be described clearly and completely below with reference to the drawings in the embodiments of the present disclosure. Obviously, the embodiments described herein are only part of the embodiments of the present disclosure, not all the embodiments. Based on the embodiments in the present disclosure, all other embodiments obtained by those of ordinary skill in the art without creative work shall fall within the protection scope of the present disclosure.

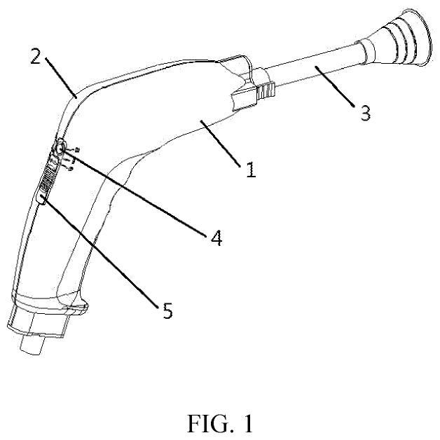

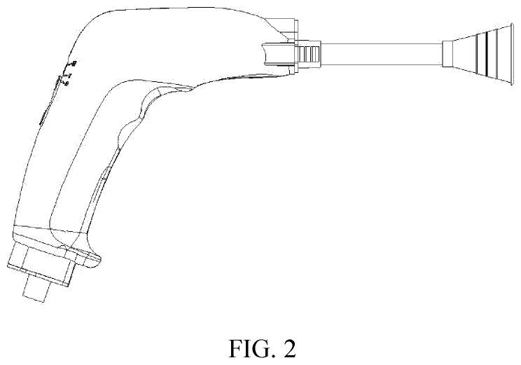

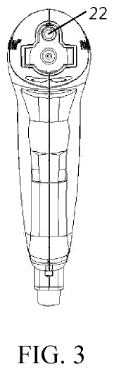

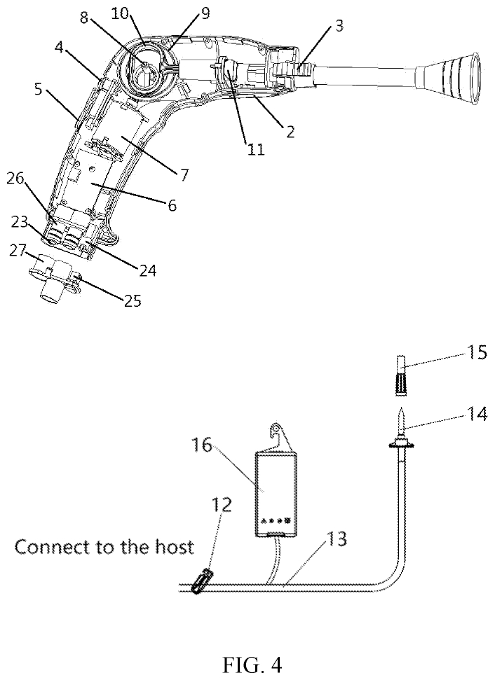

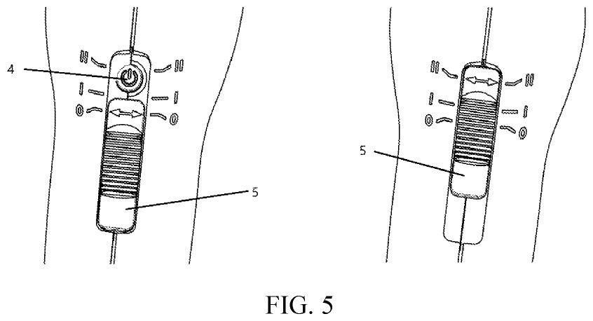

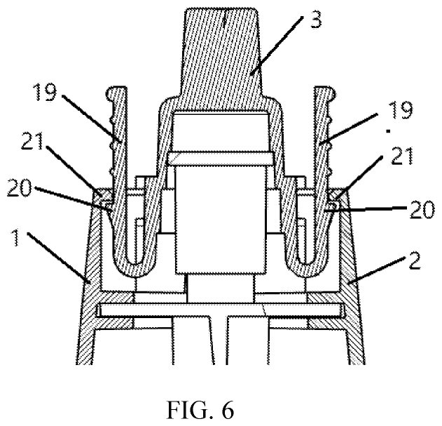

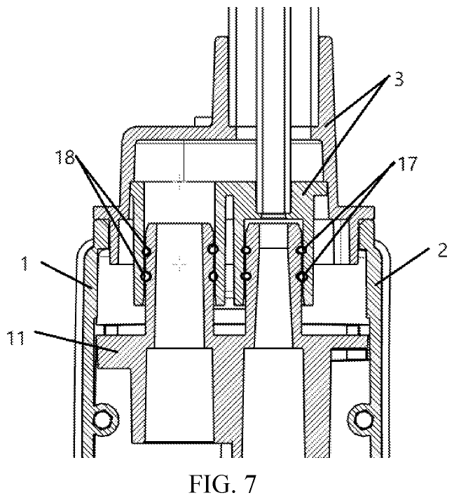

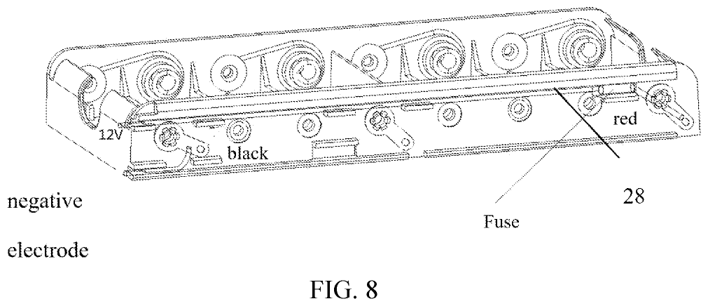

Referring to FIGS. 1 to 8, the present disclosure provides a technical solution: A stepless speed regulation medical pulse irrigator includes an upper handle (1), a lower handle (2), a spray nozzle (3), a tact switch (4), a linear speed regulation switch (5), a speed regulation control panel (6), a motor-gear assembly (7), a pin (8), a big gear (9), a piston O-ring assembly (10), a pump housing assembly (11), a clamp (12), a tube with wire (13), a spike (14), a spike cap (15), a battery pack (16), a sealing ring A (17), a sealing ring B (18), an LED lamp (22), an LED lamp control switch (23), a wire connecting body B (24), a wire connecting body A (25), a tube connecting body B (26), a tube connecting body A (27) and a battery case (28). The outer wall of the right side of the said upper handle (1) is provided with the lower handle (2), and the right side of the inner wall of the upper handle (1) is provided with the pump housing assembly (11); the left side of the said pump housing assembly (11) is connected with the piston O-ring assembly (10), and the bottom end of the piston O-ring assembly (10) is provided with the big gear (9); the pin (8) is arranged in the middle of the said big gear (9), and the bottom end of the big gear (9) is provided with the motor-gear assembly (7); the left side of the said motor-gear assembly (7) is provided with the tact switch (4); the linear speed regulation switch (5) is arranged below the said tact switch (4); the said linear speed regulation switch (5) is connected to the stepless speed regulation control panel (6) behind the motor-gear assembly (7); the said stepless speed regulation control panel (6) is connected to the motor-gear assembly (7) through two wires, and is connected to the battery pack (16) through the tube with wire (13); the said tube with wire (13) is connected to the tube connecting body A (27) and the wire connecting body A (25); the said tube connecting body B (26) and the wire connecting body B (24) are respectively connected to the tube connecting body A (27) and the wire connecting body A (25) through buckles, and are mounted in the upper handle (1) and located on two sides of the motor-gear assembly (7) and below the stepless speed regulation control panel (6); the said LED lamp control switch (23) is arranged in the tube connecting body B (26); the said tube connecting body B (26) is connected to the pump housing assembly (11) through a tube; the said clamp (12) is arranged on the tube with wire (13) at the tail part of the upper handle (1); the end part of the said tube with wire (13) is provided with the spike (14); and the top end of the said spike (14) is covered with the spike cap (15). With such technical embodiment, before irrigating a wound, the tact switch (4) will be slightly pressed; the LED indication lamp is turned on and the handpiece enters a standby state; the linear speed regulation switch (5) is slightly pushed upwards to start to irrigate the wound; the speed is slowly regulated to be appropriate according to an actual condition of the wound of a patient; and after the patient further adapts to the pressure, the speed may be regulated to location I or location II indicated on the handle to meet an irrigating pressure requirement of the wound irrigation. When irrigating fluid splashes onto a contaminated fluid collection bag to obstruct the observation, the LED lamp (22) can be turned on through the LED lamp control switch (23) to assist in the observation for the wound, which facilitates debridement personnel to determine whether debridement is completed, so that the debridement time and cost are greatly saved.

Although the embodiments of the present disclosure have been shown and described, it will be understood by those of ordinary skill in the art that various changes, modifications, substitutions, and transformations can be made to these embodiments without departing from the principle and spirit of the present disclosure. The scope of the present disclosure is defined by the attached claims and their equivalents.

What is claimed is:

1. A stepless speed regulation medical pulse irrigator with an LED lamp, comprising a upper handle (1), a lower handle (2), a spray nozzle (3), a tact switch (4), a linear speed regulation switch (5), a speed regulation control panel (6), a piston O-ring assembly (10), a pump housing assembly (11), a battery pack (16) and an LED lamp (22), wherein a right side of the said upper handle (1) is provided with the lower handle (2); the said tact switch (4) is arranged between the upper handle (1) and the lower handle (2); the linear speed regulation switch (5) is arranged below the said tact switch (4); the said spray nozzle (3) is arranged at a head part of the upper handle (1) and the lower handle (2); an outer wall of the right side of the upper handle (1) is provided with the lower handle (2), and a right side of an inner wall of the upper handle (1) is provided with the pump housing assembly (11); a left side of the pump housing assembly (11) is connected with the piston O-ring assembly (10), and a bottom end of the piston O-ring assembly (10) is provided with a big gear (9); a pin (8) is arranged in a middle of the said big gear (9), and a bottom end of the big gear (9) is provided with a motor-gear assembly (7); a left side of the said motor-gear assembly (7) is provided with the tact switch (4); the linear speed regulation switch (5) is arranged below the said tact switch (4); the linear speed regulation switch (5) is connected to the stepless speed regulation control panel (6) behind the motor-gear assembly (7); the said stepless speed regulation control panel (6) is connected to the motor-gear assembly (7) through two wires, and is connected to the battery pack (16) through a tube with wire (13); the said tube with wire (13) is connected to a tube connecting body A (27) and a wire connecting body A (25); the tube connecting body B (26) and a wire connecting body B (24) are respectively connected to the tube connecting body A (27) and the wire connecting body A (25) through buckles, and are mounted in the upper handle (1) and located on two sides of the motor-gear assembly (7) and below the stepless speed regulation control panel (6); an LED lamp control switch (23) is arranged in the tube connecting body B (26); the tube connecting body B (26) is connected to the pump housing assembly (11) through a pipeline; a clamp (12) is arranged on the tube with wire (13) at the tail part of the upper handle (1); the end part of the tube with wire (13) is provided with a spike (14); and the top end of the spike (14) is wrapped with a spike cap (15).

2. The stepless speed regulation medical pulse irrigator of claim 1, wherein the lower middle part of the inner wall in the said upper handle (1) is provided with the motor-gear assembly (7); the left side of the said motor-gear assembly (7) is provided with the tact switch (4); the linear speed regulation switch (5) is arranged below the said tact switch (4); the linear speed regulation switch (5) is connected to the stepless speed regulation control panel (6) behind the motor-gear assembly (7); the stepless speed regulation control panel (6) is connected to the motor-gear assembly (7) through two wires, and is connected to the battery pack (16) through the tube with wire (13); the rotation speed of a motor in the motor-gear assembly (7) is controlled in s stepless manner by operating the tact switch (4) and the linear speed regulation switch (5) arranged below to achieve the purpose of stepless speed regulation.

3. The stepless speed regulation medical pulse irrigator with of claim 2, wherein a self-locking structure of the said tact switch (4) and the linear speed regulation switch (5) has characteristics: the right side of the said upper handle (1) is provided with the lower handle (2); the said tact switch (4) is arranged between the upper handle (1) and the lower handle (2); the linear speed regulation switch (5) is arranged below the tact switch (4); when the switch is in an off state or a zero-speed state, the tact switch (4) is pressed, and the lamp is turned on to make the handpiece enter a standby state; at the moment, the linear speed regulation switch (5) is pushed, and the motor-gear assembly (7) starts to run; the rotation speed of the motor-gear assembly (7) also continuously increases with pushing of the linear speed regulation switch (5) till the switch is pushed to the top, and the motor-gear assembly (7) reaches the highest rotation speed; at the moment, the tact switch (4) is non-operational to achieve a self-locking purpose and ensure that the motor-gear assembly (7) runs at a rotation speed from low to high, instead of suddenly initiating the motor-gear assembly (7) at a high speed; and the experience of a patient is enhanced.

4. The stepless speed regulation medical pulse irrigator of claim 3, wherein the stepless speed regulation is carried out throughout 0-6 V-12 V, in which steady continuous operation is realized at any speed; two reference speeds are preset: a low speed (6 V) and a high speed (12 V); and a stepless speed variation module has an additional function: setting delay power cut-off, working time, the number of times, and the like, so as to enhance the experience of medical staff.

5. The stepless speed regulation medical pulse irrigator of claim 4, wherein elastic self-locking connection between the spray nozzle (3) and the upper handle (1) as well as the lower handle (2) is characterized in that: after the upper handle (1) and the lower handle (2) are correspondingly fastened, an elastic structure (19) of the spray nozzle (3) is inserted; and buckles (20) of the elastic structure (19) of the spray nozzle (3) are fastened with buckles (21) of the upper handle (1) and the lower handle (2) to complete fixing, so as to be more convenient for use and save the preparation time of instruments.

6. The stepless speed regulation medical pulse irrigator of claim 5, wherein the spray nozzle (3) is elastically hermetically connected to the pump housing assembly (11): after the upper handle (1) and the lower handle (2) are correspondingly fastened, the elastic structure (19) of the spray nozzle (3) is inserted; a sealing ring A (17) and a sealing ring B (18) close the two fluid flowing pipelines; hard sealing of a conventional product is avoided; the sealing effect is enhanced; and the machining requirement is lowered.

7. The stepless speed regulation medical pulse irrigator of claim 6, wherein the LED lamp (22) is a high-brightness white light, and the pump housing assembly (11) is in tight fit with the left side of the outer wall of the upper handle (1); the LED lamp (22), the upper handle (1) and the lower handle (2) are fixedly connected; power of the LED lamp (22) is electronically regulated from a total volume of batteries in the battery pack (16) to 3.0 to 3.6 V; two control methods for the LED lamp (22): 1, in a standby or working state, the tact switch (4) is pressed twice to turn on/off the LED lamp; and 2, in the standby or working state, the LED lamp control switch (23) is pressed once to turn on/off the LED lamp.

8. The stepless speed regulation medical pulse irrigator of claim 7, wherein the product adopts an environmentally-friendly efficient sterilization method: plasma sterilization that is short in sterilization time and effectively improves the production efficiency and reduce the cost; after the sterilization is completed, no residue is left, and the product is used immediately, which is extremely safe to human and the environment.

9. The stepless speed regulation medical pulse irrigator of claim 8, wherein a battery case (28) in the said battery pack (16) is provided with a fuse, so that a danger caused by short circuiting of the product is avoided, and the safety of the product is effectively improved.

10. The stepless speed regulation medical pulse irrigator of claim 9, wherein the said tube with wire (13) is connected with a host through tube connecting bodies (26, 27) and wire connecting bodies (24, 25); during packaging of the product, the host and pipelines are packaged separately; during use, and the host and the pipelines are connected together, improving the production process of the product and facilitating sterilization for the product; mis-initiation of the product during transportation is avoided; and product safety is improved.
